(19) United States
(12) Patent Application Publication
(10) Pub. No.: US 2022/0389493 A1
(43) Pub. Date: Dec. 8, 2022

(54) SYSTEMS AND METHODS FOR DATA STORAGE USING NUCLEIC ACID MOLECULES

(71) Applicant: Apton Biosystems, Inc., Pleasanton, CA (US)

(72) Inventors: Bryan P. STAKER, Pleasanton, CA (US); Dennis BALLINGER, Pleasanton, CA (US)

(21) Appl. No.: 17/678,264

(22) Filed: Feb. 23, 2022

Related U.S. Application Data
(63) Continuation of application No. PCT/US2020/047994, filed on Aug. 26, 2020.
(60) Provisional application No. 62/892,176, filed on Aug. 27, 2019.

(51) Int. Cl. C12Q 1/6844; C12Q 1/6869

(52) U.S. Cl. CPC C12Q 1/6844; C12Q 1/6869

(57) ABSTRACT

Disclosed herein are methods and systems for storing data and/or information on nucleic acid molecules, storing the nucleic acid molecules, and retrieving the data and/or information. These methods and systems have broad applications for data storage, including in improving the efficiency and accuracy of retrieving data.

### CROSS-REFERENCE

This application is a continuation of International Application No. PCT/US2020/047994 filed, Aug. 26, 2020, which claims benefit of U.S. Provisional Application No. 62/892,176 filed on Aug. 27, 2019, which is herein incorporated by reference in its entirety.

### BACKGROUND

The scale and complexity of the world's big data challenges and problems are rapidly growing. Meeting these challenges pose an extraordinary technological and financial hurdle. For example, exabyte-scale data storage centers are immensely resource heavy and burdensome. Current exabyte-scale data storage requires large warehouses, consumes megawatts of power, and cost billions of dollars to build, operate and maintain. This resource intensive model fails to offer a practical or feasible tractable path to scaling in the future.

### SUMMARY

The present disclosure provides methods of nucleic acid-mediated data storage that is scalable and offers a reduced resource footprint as compared to the physical space, power, and cost requirements relative to conventional storage technologies. Methods and systems described herein may provide the benefit of nuclei acid storage in which 1) arrays can be generated in ready-to-read manner wherein no amplification of a nucleic acid sequence prior to sequencing/reading and 2) nucleic acids encoding data information can be stored on high density arrays at densities wherein the distance between one or more nucleic acid molecules is below the diffraction limit of light.

An aspect of the disclosure described herein provides a method for storing data, comprising: encoding said data in a nucleic acid sequence; generating one or more nucleic acid molecules, wherein a nucleic acid molecule of said one or more nucleic acid molecules comprises at least a portion of said nucleic acid sequence and a header sequence, wherein said header sequence comprises a sequence that is specific to said at least said portion of said nucleic acid sequence, and wherein said header sequence is configured to permit initiation of a nucleic acid identification reaction for identifying said at least said portion of said nucleic acid sequence; and storing said one or more nucleic acid molecules or derivative thereof in an array disposed on a substrate. In some embodiments, said nucleic acid identification reaction is a sequencing reaction. In some embodiments, said one or more nucleic acid molecules or derivative thereof are linear. In some embodiments, the method further comprises preserving said one or more nucleic acid molecules or derivative thereof. In some embodiments, said preserving comprises lyophilization or freeze-drying. In some embodiments, (b) further comprises amplifying said at least said portion of said nucleic acid sequence to form one or more amplification products, wherein said one or more nucleic acid molecules comprise said one or more amplification products. In some embodiments, said amplifying comprises performing rolling circle amplification. In some embodiments, said amplifying comprises performing bridge amplification. In some embodiments, said one or more nucleic acid molecules or derivative thereof comprise concatenated nucleic acid molecules. In some embodiments, said one or more nucleic acid molecules or derivative thereof are disposed on said substrate at a density wherein a distance between a nucleic acid molecule or derivative thereof of said one or more nucleic acid molecules or derivative thereof and an adjacent nucleic acid molecule or derivative thereof is less than 500 nm. In some embodiments, said distance comprises a center-to-center distance. In some embodiments, said one or more nucleic acid molecules or derivative thereof are disposed on said substrate at a density of about 4 to about 25 nucleic acid molecules or derivative thereof per square micron. In some embodiments, the method further comprises retrieving said data. In some embodiments, said retrieving comprises sequencing said one or more nucleic acid molecules or derivative thereof. In some embodiments, said sequencing comprises detecting one or more incorporated nucleic acids using detection system. In some embodiments, said detection system comprises an electrical detection system. In some embodiments, said electrical detection system comprises a transistor. In some embodiments, said detection system comprises an optical detection system. In some embodiments, said optical detection system comprises an optical scanning system. In some embodiments, a wavelength of a signal generated from said one or more incorporated nucleic acids detected on said optical detection system is greater than two times a pixel of said optical detection system. In some embodiments, said array is ordered. In some embodiments, said array is nonordered. In some embodiments, said start site comprises a nucleic acid sequence complementary to a nucleic acid primer. In some embodiments, said amplifying occurs prior to said storing.

Another aspect of the disclosure described herein provides a method for storing data, comprising: encoding said data in a nucleic acid sequence; generating one or more nucleic acid molecules comprising said nucleic acid sequence; and storing said one or more nucleic acid molecules in an array disposed on a substrate, to provide said array wherein when said array is imaged using an optical scanning system, a wavelength of a signal generated from said one or more nucleic acid molecules or derivative thereof is greater than two times a size of a pixel of said optical scanning system. In some embodiments, said one or more nucleic acid molecules are linear. In some embodiments, (b) comprises generating one or more linear nucleic acid molecules comprising at least a portion of said nucleic acid sequence and circularizing said one or more linear nucleic acid molecules and amplifying by rolling circle amplification to generate one or more concatenated nucleic acid molecules. In some embodiments, (b) comprises generating one or more linear nucleic acid molecules that comprise said nucleic acid sequence, a first adapter sequence, and a second adapter sequence, wherein said first and said second adapter sequence enable formation of one or more circular nucleic acid molecules; and amplifying said one or more circular nucleic acid molecules. In some embodiments, said linear nucleic acid molecule comprises one or more functional sequences. In some embodiments, said one or more concatemeric nucleic acid molecules are generated by a rolling circle amplification. In some embodiments, (c) comprises disposing said concatemeric nucleic acid molecules on said substrate. In some embodiments, said one or more concatemeric nucleic acid molecules are disposed at a density wherein an average distance between two or more nucleic acid molecules is less than a measure of λ/(2*NA). In some embodiments, the method further comprises preserving said substrate. In some embodiments, said preserving comprises lyophilization or freeze-drying. In some embodiments, said substrate comprises silicon. In some embodiments, said substrate comprises glass. In some embodiments, said substrate comprises two pieces of glass. In some embodiments, the method further comprises retrieving said data from said one or more nucleic acid molecules without amplification prior to said retrieving. In some embodiments, said array is ordered. In some embodiments, said array is nonordered. In some embodiments, said order is random.

Another aspect of the disclosure described herein provides a method for storing data, comprising disposing a nucleic acid molecule to a substrate, wherein said nucleic molecule or derivative thereof encodes said data. In some embodiments, said nucleic acid molecule or derivative thereof comprises a nucleic acid concatemer. In some embodiments, said nucleic acid molecule or derivative thereof is disposed at a density wherein when said substrate is imaged using an optical scanning system, a wavelength of a signal generated from said nucleic acid molecule or derivative thereof is greater than two times a size of a pixel of said optical scanning system. In some embodiments, said substrate comprises silicon. In some embodiments, said substrate comprises glass. In some embodiments, said substrate comprises two pieces of glass. In some embodiments, said data is retrieved from said nucleic acid molecule without amplification prior to sequencing.

Another aspect of the disclosure described herein provides a method of storing one or more bits of information, said method comprising: encoding said one or more bits of information in a plurality of nucleotides; coupling said plurality of nucleotides to one or more primers; synthesizing said plurality of nucleotides to a length of about 300 to about 1,000 nucleotides; circularizing said plurality of nucleotides; amplifying said plurality of circular molecules by rolling circle amplification to generate one or more nucleic acid molecules; and disposing said one or more nucleic acid molecules onto a substrate.

Another aspect of the disclosure described herein provides a method of storing one or more bits of information, said method comprising: synthesizing a linear nucleic acid molecule that encodes said one or more bits of information, wherein said linear nucleic acid molecule comprises: a nucleic acid sequence that encodes said one or more bits of information, a 5′ adapter sequence, a 3′ adapter sequence, and an optional one or more additional functional sequences, generating a circular nucleic acid molecule from said linear nucleic acid molecule, amplifying said circular nucleic acid molecule to generate an amplified nucleic acid molecule that comprises more than one copy of said circular nucleic acid molecule, disposing said amplified nucleic acid molecule on a substrate. In some embodiments, said substrate is patterned. In some embodiments, said substrate is unpatterned. In some embodiments, the method further comprises preserving said one or more substrates. In some embodiments, said preserving comprises lyophilization or freeze-drying. In some embodiments, the method further comprises retrieving said one or more bits of information from said one or more nucleic acid molecules without amplification prior to said retrieving. In some embodiments, said retrieving said one or more bits of information comprises a nucleic acid identification reaction. In some embodiments, the method further comprises applying an error correction to a recovered one or more bits of information. In some embodiments, said error correction comprises using a Reed-Solomon code. In some embodiments, said bits of information comprise binary bits. In some embodiments, said bits of information comprise binary bits and (a) comprises transcribing said binary bits of information into quaternary bits of information. In some embodiments, said 5′ adapter sequence, 3′ adapter sequence, or both comprise a barcode sequence. In some embodiments, said one or more functional sequences is selected from the group consisting of a barcode sequence, a tag sequence, a universal primer sequence, a unique identifier sequence, or an additional adapter sequence. In some embodiments, said circular nucleic molecule is generated by ligating said 5′ adapter and said 3′ adapter. In some embodiments, said circular nucleic molecule is amplified by a rolling circle reaction. In some embodiments, said amplified nucleic acid molecule is a nucleic acid concatemer. In some embodiments, said amplified nucleic acid molecule is disposed at a density wherein when said substrate is imaged using an optical scanning system, a wavelength of a signal generated from said nucleic acid molecule or derivative thereof is greater than two times a size of a pixel of said optical scanning system. In some embodiments, said substrate comprises silicon. In some embodiments, said substrate comprises glass. The method of any one of the preceding embodiments, wherein said array comprises a first and a second glass substrate. The method of any one of the preceding embodiments, wherein the method is automated by a computer system that is programmed to implement a method as in any one of the preceding embodiments.

Another aspect of the disclosure described herein provides a computer system, wherein the computer system is programmed to implement a method as in any one of the preceding embodiments.

Another aspect of the disclosure described herein provides a nucleic acid molecule comprising a plurality of nucleic acid sequences, wherein at least a portion said plurality of nucleic acid sequences encode at least 1 gigabytes (GB) of data, and wherein said nucleic acid molecule has a stability such that no more than 1% of said nucleic acid molecule degrades over a period of 1 year. The nucleic acid molecule of the preceding embodiment, further comprising a plurality of header sequences, wherein a header sequence of said plurality of header sequences is configured to permit sequencing of at least said portion of said nucleic acid sequence to retrieve said 1 GB of data.

Another aspect of the disclosure described herein provides a method for storing data, comprising (a) encoding said data in a nucleic acid sequence; (b) generating one or more nucleic acid molecules comprising said nucleic acid sequence; and (c) storing said one or more nucleic acid molecules in an array disposed on a substrate. In some embodiments, said one or more nucleic acid molecules are circular. In some embodiments, (b) comprises generating one or more circular nucleic acid molecules comprising at least a portion of said nucleic acid sequence and amplifying said one or more circular nucleic acid molecules by rolling circle amplification to generate one or more concatenated copies of individual nucleic acid molecules. In some embodiments, (b) comprises generating one or more linear nucleic acid molecules that comprise said nucleic acid sequence, a first adapter sequence, and a second adapter sequence, wherein said first and said second adapter sequence enable formation of one or more circular nucleic acid molecules; and amplifying said one or more circular nucleic acid molecules. In some embodiments, said linear nucleic acid molecule comprises one or more functional sequences. In some embodiments, one or more concatenated nucleic acid molecules are amplified by a rolling circle amplification. In some embodiments, (c) comprises disposing said concatenated copies of nucleic acid molecules on said substrate. In some embodiments, said one or more concatenated nucleic acid molecules are disposed at a density wherein an average distance between two or more nucleic acid molecules is less than a measure of λ/(2*NA). In some embodiments, the method further comprises preserving said substrate. In some embodiments, said preserving comprises lyophilization or freeze-drying. In some embodiments, said substrate comprises silicon. In some embodiments, said substrate comprises glass. In some embodiments, said substrate comprises two pieces of glass. In some embodiments, the method further comprises retrieving said data from said one or more nucleic acid molecules without amplification prior to said retrieving.

Another aspect described herein provides a method for storing data, comprising disposing a nucleic acid molecule to a substrate, wherein said nucleic molecule encodes said data. In some embodiments, said nucleic acid molecule comprises a nucleic acid concatemer. In some embodiments, said concatemer molecules are disposed at a density wherein an average distance between a first and a second circular nucleic acid molecule is less than a measure of λ/(2*NA). In some embodiments, said substrate comprises silicon. In some embodiments, said substrate comprises glass. In some embodiments, said substrate comprises two pieces of glass. In some embodiments, said data is retrieved from nucleic acid molecule without circularization or amplification prior to sequencing.

Another aspect described herein provides a method of storing one or more bits of information, said method comprising: encoding said one or more bits of information in a plurality of nucleotides; coupling said plurality of nucleotides to one or more primers; synthesizing said plurality of nucleotides to a range of about 300 to about 1,000 nucleotides; circularizing said plurality of nucleotides, and disposing said plurality of nucleotides onto a substrate.

Another aspect described herein provides method of storing one or more bits of information, said method comprising: synthesizing a linear nucleic acid molecule that encodes said one or more bits of information, wherein said linear nucleic acid molecule comprises: a nucleic acid sequence that encodes said one or more bits of information, a 5′ adapter sequence, a 3′ adapter sequence, and an optional one or more additional functional sequences, generating a circular nucleic molecule from said linear nucleic acid molecule, amplifying said circular nucleic acid molecule to generate an second nucleic acid molecule that comprises more than one copy of the circular nucleic acid molecule, disposing said second nucleic acid molecule on an array. In some embodiments, the method further comprises disposing said array on to one or more substrates. In some embodiments, the method further comprises preserving said one or more substrates. In some embodiments, said preserving comprises lyophilization or freeze-drying. In some embodiments, the method further comprises retrieving said one or more bits of information from said one or more nucleic acid molecules without amplification prior to said retrieving. In some embodiments, said one or more bits of information is recovered from said array by a sequencing reaction. In some embodiments, the method further comprises applying an error correction to a recovered one or more bits of information. In some embodiments, said error correction comprises using a Reed-Solomon code. In some embodiments, said one or more bits of information is retrieved from said array without an amplification replication reaction prior to sequencing. In some embodiments, said bits of information comprise binary bits. In some embodiments, said bits of information comprise binary bits and (a) comprises transcribing said binary bits of information into quaternary bits of information. In some embodiments, said adapter sequence comprises a barcode sequence. In some embodiments, said one or more functional sequences is selected from the group consisting of a barcode sequence, a tag sequence, a universal primer sequence, a unique identifier sequence, or an additional adapter sequence. In some embodiments, said circular nucleic molecule is generated by ligating said 5′ adapter and said 3′ adapter. In some embodiments, said circular nucleic molecule is amplified by a rolling circle PCR reaction. In some embodiments, said second nucleic acid molecule is a nucleic acid concatemer. In some embodiments, said second nucleic acid molecule is disposed at a density wherein an average distance between two or more nucleic acid molecules is less than a measure of λ/(2*NA). In some embodiments, said array comprises a siliconized substrate. In some embodiments, said array comprises a glass substrate. In some embodiments, said array comprises a first and a second glass substrate. In some embodiments, the method is automated by a computer system that is programmed to implement a method as in any one of the preceding claims.

Another aspect described herein provides a computer system, wherein the computer system is programmed to implement a method as described herein.

Another aspect described herein provides a plurality of nucleic acid molecules comprising a nucleic acid sequence at least a portion of which encodes at least 1 gigabytes (GB) of data, wherein said nucleic acid molecules have a stability such that no more than 1% of said nucleic acid sequence degrades over a period of 1 year. In some embodiments, the nucleic acid molecules are circular. In some embodiments, the nucleic acid molecules further comprise a plurality of header sequences, wherein a header sequence of said plurality of header sequences is configured to permit sequencing of said at least said portion of said nucleic acid sequence to retrieve said 1 GB of data.

Another aspect described herein provides a method for storing data, comprising (a) encoding the data in a nucleic acid sequence; (b) generating a nucleic acid molecule comprising the nucleic acid sequence; and (c) storing the nucleic acid molecule on an array. In some embodiments, the nucleic acid molecule is circular. In some embodiments, the nucleic acid molecule is a nucleic acid concatemer. In some embodiments, (b) comprises generating a linear nucleic acid molecule comprising at least a portion of the nucleic acid sequence, and coupling ends of the linear nucleic acid molecules to one another to generate a circular nucleic acid molecule. In another embodiment (b) comprises (i) generating a linear nucleic acid molecule that comprises the linear nucleic acid molecule, a first adapter sequence, and a second adapter sequence, wherein the first and the second adapter sequence enable formation of the circular nucleic acid molecule; and (ii) amplifying the circular nucleic acid molecule to generate a nucleic acid concatemer. In some embodiments, the linear nucleic acid molecule comprises a functional sequence. In some embodiments, the linear nucleic acid molecule comprises a plurality of functional sequences.

In some embodiments, the nucleic acid concatemer is generated by a rolling circle amplification. In some embodiments, (c) comprises disposing the nucleic acid molecule on a substrate. In some embodiments, the nucleic acid molecule is disposed at a density wherein an average distance between two or more nucleic acid molecules is less than a measure of λ/(2*NA). In some embodiments, the array comprises a silicon substrate. In some embodiments the array comprises a glass substrate.

In some embodiments, the data is retrieved from nucleic acid molecule without polymerase chain reaction amplification prior to sequencing.

In another aspect, disclosed is a method for storing data, comprising immobilizing or disposing a nucleic acid molecule to a substrate, wherein the nucleic molecule encodes the data. In some embodiments, the nucleic acid molecule comprises a nucleic acid concatemer. In some embodiments the nucleic acid molecule is immobilized or disposed at a density wherein an average distance between a first and a second nucleic acid molecule is less than a measure of λ/(2*NA). In some embodiments the substrate comprises silicon. In some embodiments the substrate comprises glass. In some embodiments the data is retrieved from nucleic acid molecule without amplification prior to sequencing.

In another aspect, disclosed is a method of storing one or more bits of information, the method comprising (a) encoding the one or more bits of information in a plurality of nucleotides, (b) coupling the plurality of nucleotides to one or more primers, (c) synthesizing the plurality of nucleotides to a range of about 300 to about 1,000 nucleotides, (d) circularizing the plurality of nucleotides, and (e) disposing the plurality of nucleotides onto a substrate.

In another aspect, disclosed is a method of storing one or more bits of information, the method comprising (a) synthesizing a linear nucleic acid molecule that encodes the one or more bits of information, wherein the linear nucleic acid molecule comprises (i) a nucleic acid sequence that encodes the data, (ii) a 5′ adapter sequence, (iii) a 3′ adapter sequence, and (iv) an optional one or more additional functional sequences, and (b) generating a circular nucleic molecule from the linear nucleic acid molecule, and (c) amplifying the circular nucleic acid molecule to generate an second nucleic acid molecule that comprises more than one copy of the circular nucleic acid molecule, and (d) immobilizing or disposing the second nucleic acid molecule on a patterned or unpatterned array.

In some embodiments the information is recovered from the array by a sequencing reaction. In some embodiments, recovering the information further comprises applying an error correction to a recovered one or more bits of information. In some embodiments, the error correction comprises using a Reed-Solomon code. In some embodiments the information is retrieved from the array without an amplification replication reaction prior to sequencing.

In some embodiments, the bits of information comprise binary bits. In some embodiments the bits of information comprise binary bits and (a) comprises transcribing the binary bits of information into quaternary bits of information. In some embodiments the adapter sequence comprises a barcode sequence. the one or more functional sequences is selected from the group consisting of a barcode sequence, a tag sequence, a universal primer sequence, a unique identifier sequence, or an additional adapter sequence. In some embodiments, the circular nucleic molecule is generated by ligating the 5′ adapter and the 3′ adapter. In some embodiments, the circular nucleic molecule is amplified by a rolling circle reaction. In some embodiments, the second nucleic acid molecule is a nucleic acid concatemer. In some embodiments, the second nucleic acid molecule is immobilized or disposed on the substrate at a density wherein an average distance between two or more nucleic acid molecules is less than a measure of λ/(2*NA).

In some embodiments, the array comprises a siliconized substrate. In some embodiments the array comprises a glass substrate. In some embodiments the array comprises a first and a second glass substrate.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts a schematic for encoding bits of information or data in a nucleic acid molecule and disposing the nucleic acid molecule on an array. The array is then disposed onto a substrate and either stored for long-term storage, sequenced, or stored and then sequenced.

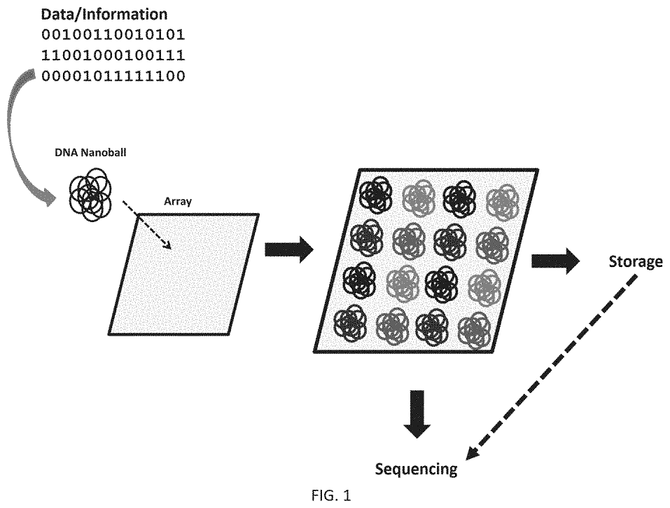

FIG. 2 depicts a schematic for utilizing a computer system to automate the systems and methods described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “concatemer” refers to a copy of a circular nucleic acid molecule. Concatemers may be generated from circular nucleic acid molecules that are amplified by rolling circle amplification after the ends of a linear nucleic acid molecule are ligated to achieve circular nucleic acid molecule. Concatemers can contain a single sequence of nucleic acids that repeat throughout the entire molecule, or they can contain different sequences of nucleic acid sequences wherein each distinct sequence or set of repeated sequences are separated by adapter sequences or regions.

As used herein, “instruments for sequencing” refers to instruments, including hardware, software, reagents, imaging modules, and/or any combination thereof familiar to those with ordinary skill in the art of nucleic acid molecule sequencing.

As used herein, “analytes” refer to any one or more molecules suitable for analysis.

Including, but not limited to, nucleic acid molecules, proteins, peptides, etc. Throughout the disclosure described herein, the term “analyte(s)” can be used inter-changeably with “nucleic acid(s)” and/or “nucleic acid molecule(s)” and/or “circular nucleic acid molecule(s)” and/or concatemers without changing the scope of the disclosure.

As used herein, “header sequence(s)” refer to known sequences addressable with distinct sequencing primers.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In a case, the method comprises storing data, comprising (a) encoding the data in a nucleic acid sequence; (b) generating a nucleic acid molecule comprising the nucleic acid sequence; and (c) storing the nucleic acid molecule analyte on an ordered or unordered array. In an instance, the nucleic acid molecule is circular. In an instance, the nucleic acid molecule is a nucleic acid concatemer. In an instance, (b) comprises generating a linear nucleic acid molecule comprising at least a portion of the nucleic acid sequence, and coupling ends of the linear nucleic acid molecules to one another to generate the circular nucleic acid molecule. In another instance (b) comprises (i) generating a linear nucleic acid molecule that comprises the linear nucleic acid molecule, a first adapter sequence, and a second adapter sequence, wherein the first and the second adapter sequence enable formation of the circular nucleic acid molecule; and (ii) amplifying the circular nucleic acid molecule to generate a nucleic acid concatemer. In some instances, the linear nucleic acid molecule comprises a functional sequence. In some instances, the linear nucleic acid molecule comprises a plurality of functional sequences.

In an instance, the nucleic acid concatemer is generated by a rolling circle amplification. In an instance, (c) comprises disposing the analyte nucleic acid molecule on a substrate. In some instances, the analyte is disposed at a density wherein an average distance between two or more nucleic acid molecules is less than a measure of λ/(2*NA). In some instances, the array comprises a silicon substrate. In some instances the array comprises a glass substrate.

In an instance, the data is retrieved from nucleic acid molecule without amplification prior to sequencing.

In a case, disclosed is a method for storing data, comprising immobilizing or disposing a nucleic acid molecule to a substrate, wherein the nucleic molecule encodes the data. In an instance, the nucleic acid molecule comprises a nucleic acid concatemer. In an instance the circular nucleic acid molecule is immobilized or disposed at a density wherein an average distance between a first and a second circular nucleic acid molecule is less than a measure of λ/(2*NA). In some instances the substrate comprises silicon. In some instances the substrate comprises glass. In an instance the data is retrieved from nucleic acid molecule without polymerase chain reaction amplification prior to sequencing.

In a case, the method comprises storing one or more bits of information, the method comprising (a) encoding the one or more bits of information in a plurality of nucleotides, (b) coupling the plurality of nucleotides to one or more primers, (c) synthesizing the plurality of nucleotides to a range of about 300 to about 1,000 nucleotides, (d) circularizing (or not) the plurality of analytes, and (e) disposing the plurality of analytes onto a substrate.

In a fourth case, the method comprises storing one or more bits of information, the method comprising (a) synthesizing a linear nucleic acid molecule that encodes the one or more bits of information, wherein the linear nucleic acid molecule comprises (i) a nucleic acid sequence that encodes the data, (ii) a 5′ adapter sequence, (iii) a 3′ adapter sequence, and (iv) an optional one or more additional functional sequences, and (b) generating a circular nucleic molecule from the linear nucleic acid molecule, and (c) amplifying the circular nucleic acid molecule to generate an analyte that comprises more than one copy of the circular nucleic acid molecule, and (d) immobilizing or disposing the analyte on an array.

In an instance the information is recovered from the array by a sequencing reaction. In an instance, recovering the information further comprises applying an error correction to a recovered one or bits of information. In some instances, the error correction comprises using a Reed-Solomon code. In an instance the information is retrieved from the array without an amplification replication reaction prior to sequencing.

In an instance, the bits of information comprise binary bits. In an instance the bits of information comprise binary bits and (a) comprises transcribing the binary bits of information into quaternary bits of information. In an instance the adapter sequence comprises a barcode sequence. the one or more functional sequences is selected from the group consisting of a barcode sequence, a tag sequence, a universal primer sequence, a unique identifier sequence, or an additional adapter sequence. In an instance, the circular nucleic molecule is generated by ligating the 5′ adapter and the 3′ adapter. In an instance, the circular nucleic molecule is amplified by a rolling circle PCR reaction. In an instance, the second nucleic acid molecule is a nucleic acid concatemer. In an instance, the second nucleic acid molecule is disposed at a density wherein an average distance between two or more nucleic acid molecules is less than a measure of λ/(2*NA).

In an instance, the array comprises a siliconized substrate. In an instance the array comprises a glass substrate. In an instance the array comprises a first and a second glass substrate.

Sequencing technologies include image based systems developed by companies such as Illumina and Complete Genomics and electrical based systems developed by companies such as Ion Torrent and Oxford Nanopore. Image based sequencing systems currently have the lowest sequencing costs of all existing sequencing technologies. Image based systems achieve low cost through the combination of high throughput imaging optics and low cost consumables. However, prior art optical detection systems have minimum center-to-center spacing between adjacent resolvable molecules at about a micron, in part due to the diffraction limit of optical systems. In some embodiments, described herein are methods for attaining significantly lower costs for an image based sequencing system using existing biochemistries using cycled detection, determination of precise positions of analytes, and use of the positional information for highly accurate deconvolution of imaged signals to accommodate increased packing densities that operate below the diffraction limit.

### Disposing Nucleic Acid Molecules on Substrate for Long-Term Storage

Provided herein are systems and methods for storing information on encoded nucleic acid molecules and processing the nucleic acid molecules for long-term storage. The systems and methods described herein are directed to processing techniques that preserve the nucleic acid molecules such that the nucleic acid molecules either do not degrade or degrade at a commercially viable rate.

In some embodiments, the nucleic acid molecules are processed either as a single segment or a series of segments comprising the stored information segments and necessary information (e.g. Reed-Solomon codes or redundancy) to ensure rapid and accurate retrieval. The segment length for the nucleic acid molecules are chosen to ensure both the accurate synthesis (by sequencing-by-synthesis techniques or other sequencing approaches) and accurate retrieval by sequencing technology and instrument(s). In some embodiments, information segments are in the range of 50-75 bases are appropriately sized for both synthesis and retrieval.

In some embodiments, the information segments are in the length of about 30 bases to about 140 bases. In some embodiments, the information segments are in the length of about 30 bases to about 40 bases, about 30 bases to about 50 bases, about 30 bases to about 60 bases, about 30 bases to about 70 bases, about 30 bases to about 80 bases, about 30 bases to about 90 bases, about 30 bases to about 100 bases, about 30 bases to about 110 bases, about 30 bases to about 120 bases, about 30 bases to about 130 bases, about 30 bases to about 140 bases, about 40 bases to about 50 bases, about 40 bases to about 60 bases, about 40 bases to about 70 bases, about 40 bases to about 80 bases, about 40 bases to about 90 bases, about 40 bases to about 100 bases, about 40 bases to about 110 bases, about 40 bases to about 120 bases, about 40 bases to about 130 bases, about 40 bases to about 140 bases, about 50 bases to about 60 bases, about 50 bases to about 70 bases, about 50 bases to about 80 bases, about 50 bases to about 90 bases, about 50 bases to about 100 bases, about 50 bases to about 110 bases, about 50 bases to about 120 bases, about 50 bases to about 130 bases, about 50 bases to about 140 bases, about 60 bases to about 70 bases, about 60 bases to about 80 bases, about 60 bases to about 90 bases, about 60 bases to about 100 bases, about 60 bases to about 110 bases, about 60 bases to about 120 bases, about 60 bases to about 130 bases, about 60 bases to about 140 bases, about 70 bases to about 80 bases, about 70 bases to about 90 bases, about 70 bases to about 100 bases, about 70 bases to about 110 bases, about 70 bases to about 120 bases, about 70 bases to about 130 bases, about 70 bases to about 140 bases, about 80 bases to about 90 bases, about 80 bases to about 100 bases, about 80 bases to about 110 bases, about 80 bases to about 120 bases, about 80 bases to about 130 bases, about 80 bases to about 140 bases, about 90 bases to about 100 bases, about 90 bases to about 110 bases, about 90 bases to about 120 bases, about 90 bases to about 130 bases, about 90 bases to about 140 bases, about 100 bases to about 110 bases, about 100 bases to about 120 bases, about 100 bases to about 130 bases, about 100 bases to about 140 bases, about 110 bases to about 120 bases, about 110 bases to about 130 bases, about 110 bases to about 140 bases, about 120 bases to about 130 bases, about 120 bases to about 140 bases, or about 130 bases to about 140 bases. In some embodiments, the information segments are in the length of about 30 bases, about 40 bases, about 50 bases, about 60 bases, about 70 bases, about 80 bases, about 90 bases, about 100 bases, about 110 bases, about 120 bases, about 130 bases, or about 140 bases. In some embodiments, the information segments are in the length of at least about 30 bases, about 40 bases, about 50 bases, about 60 bases, about 70 bases, about 80 bases, about 90 bases, about 100 bases, about 110 bases, about 120 bases, or about 130 bases. In some embodiments, the information segments are in the length of at most about 40 bases, about 50 bases, about 60 bases, about 70 bases, about 80 bases, about 90 bases, about 100 bases, about 110 bases, about 120 bases, about 130 bases, or about 140 bases.

In some embodiments, the nucleic acid molecules are attached to appropriate adapters for subsequent conversion to circular nucleic acid molecules (e.g. CATs or concatemers), for example, by rolling circle amplification, and attachment to appropriate substrates for sequencing and detection (as per US20150330974 or US20160201119 and/or U.S. Pat. No. 10,378,053). Common sequences minimally contain sequences appropriate for the priming of sequencing and circularization the nucleic acid molecules. In some embodiments, the full length of the circularized nucleic acid molecules is in the range of 300-1,000 bases. In some embodiments, the length of the circularized nucleic acid molecules could be achieved by appending multiple information segments within the same circle, separated by sequences addressable with different sequencing primers (referred to as “header sequences” herein). In some embodiments, the length of the circularized nucleic acid molecules could be achieved by introducing stuffer fragments that would not be sequenced to achieve the appropriate size.

In some embodiments, the length of the circularized nucleic acid molecules is about 200 bases to about 1,200 bases. In some embodiments, the length of the circularized nucleic acid molecules are about 200 bases to about 300 bases, about 200 bases to about 400 bases, about 200 bases to about 500 bases, about 200 bases to about 600 bases, about 200 bases to about 700 bases, about 200 bases to about 800 bases, about 200 bases to about 900 bases, about 200 bases to about 1,000 bases, about 200 bases to about 1,100 bases, about 200 bases to about 1,200 bases, about 300 bases to about 400 bases, about 300 bases to about 500 bases, about 300 bases to about 600 bases, about 300 bases to about 700 bases, about 300 bases to about 800 bases, about 300 bases to about 900 bases, about 300 bases to about 1,000 bases, about 300 bases to about 1,100 bases, about 300 bases to about 1,200 bases, about 400 bases to about 500 bases, about 400 bases to about 600 bases, about 400 bases to about 700 bases, about 400 bases to about 800 bases, about 400 bases to about 900 bases, about 400 bases to about 1,000 bases, about 400 bases to about 1,100 bases, about 400 bases to about 1,200 bases, about 500 bases to about 600 bases, about 500 bases to about 700 bases, about 500 bases to about 800 bases, about 500 bases to about 900 bases, about 500 bases to about 1,000 bases, about 500 bases to about 1,100 bases, about 500 bases to about 1,200 bases, about 600 bases to about 700 bases, about 600 bases to about 800 bases, about 600 bases to about 900 bases, about 600 bases to about 1,000 bases, about 600 bases to about 1,100 bases, about 600 bases to about 1,200 bases, about 700 bases to about 800 bases, about 700 bases to about 900 bases, about 700 bases to about 1,000 bases, about 700 bases to about 1,100 bases, about 700 bases to about 1,200 bases, about 800 bases to about 900 bases, about 800 bases to about 1,000 bases, about 800 bases to about 1,100 bases, about 800 bases to about 1,200 bases, about 900 bases to about 1,000 bases, about 900 bases to about 1,100 bases, about 900 bases to about 1,200 bases, about 1,000 bases to about 1,100 bases, about 1,000 bases to about 1,200 bases, or about 1,100 bases to about 1,200 bases. In some embodiments, the length of the circularized nucleic acid molecules are about 200 bases, about 300 bases, about 400 bases, about 500 bases, about 600 bases, about 700 bases, about 800 bases, about 900 bases, about 1,000 bases, about 1,100 bases, or about 1,200 bases. In some embodiments, the length of the circularized nucleic acid molecules are at least about 200 bases, about 300 bases, about 400 bases, about 500 bases, about 600 bases, about 700 bases, about 800 bases, about 900 bases, about 1,000 bases, or about 1,100 bases. In some embodiments, the length of the circularized nucleic acid molecules is at most about 300 bases, about 400 bases, about 500 bases, about 600 bases, about 700 bases, about 800 bases, about 900 bases, about 1,000 bases, about 1,100 bases, or about 1,200 bases.

In some embodiments, the circular nucleic acid molecules are disposed onto a substrate (such as a chip for sequencing). In some embodiments, after one or more nucleic acid molecules are disposed onto a substrate, the substrate will have to be processed for long-term storage. In some embodiments, the process comprises drying the substrate. In some embodiments, the process comprises freeze drying, such as by lyophilization or cryodesiccation. Lyophilization may include use of a freeze-drying process comprising a low temperature dehydration process which may involve freezing a product, lowering pressure, then removing the ice by sublimation. In some embodiments, prior to the drying process, the substrate disposed with the circular nucleic acid molecules is treated (as post-load treatments) to ensure stability during and recovery from the drying process. In some embodiments, the treatments comprise coating the surface of the substrate with e.g., BSA or Dextran Sulfate to stabilize the circular nucleic acid molecules as well as the introduction of appropriate excipients such as sugars (e.g., mannitol, sucrose, trehalose, lactose, maltose, glucose, glycine, glycerol, etc.) and appropriate buffers to stabilize and protect the substrate from ice crystal formation during the freeze-drying, and shock during re-hydration.

In some embodiments, amplification of the nucleic acid molecules (e.g. rolling circle amplification) occurs prior to long-term storage of the substrate(s) comprising the nucleic acid molecules. In some embodiments, amplification of the nucleic acid molecules occurs on the substrate which the nucleic acid molecules are disposed on. In some embodiments, the amplification is bridge amplification. In some embodiments, amplification of the nucleic acid molecules (e.g. rolling circle amplification) occurs prior to disposing the nucleic acid molecules on the substrate. In some embodiments, the amplification is rolling circle amplification.

In some embodiments, the circular nucleic acid molecules are disposed onto a plurality slides for storage. In some embodiments, the slides have a plurality of distinct lanes and/or tracks. In some embodiments, the unique header sequences are used to identify positional information for a specific sequence comprising information. In some embodiments, the positional information is found in a catalog comprising information for every header sequence used to store a given set of information. In some embodiments, while the information set up for eventual retrieval is contained in nucleic acid molecules disposed on the substrate/slides for storage, a plurality of copies of the nucleic acid molecules are stored separately as back-up information. In some embodiments, in addition to future-proofing the information storage process, the nucleic acid molecules corresponding to each lane are separately dried and stored as a back-up. In some embodiments, the back-up nucleic acid molecules can be subsequently processed as appropriate in the event the information on the originally processed stored slides is irretrievable.

In some embodiments, degradation rate of the preserved nucleic acids is about 0.05% per year to about 2% per year. In some embodiments, degradation rate of the preserved nucleic acids is about 2% per year to about 1% per year, about 2% per year to about 0.9% per year, about 2% per year to about 0.8% per year, about 2% per year to about 0.7% per year, about 2% per year to about 0.6% per year, about 2% per year to about 0.5% per year, about 2% per year to about 0.4% per year, about 2% per year to about 0.3% per year, about 2% per year to about 0.2% per year, about 2% per year to about 0.1% per year, about 2% per year to about 0.05% per year, about 1% per year to about 0.9% per year, about 1% per year to about 0.8% per year, about 1% per year to about 0.7% per year, about 1% per year to about 0.6% per year, about 1% per year to about 0.5% per year, about 1% per year to about 0.4% per year, about 1% per year to about 0.3% per year, about 1% per year to about 0.2% per year, about 1% per year to about 0.1% per year, about 1% per year to about 0.05% per year, about 0.9% per year to about 0.8% per year, about 0.9% per year to about 0.7% per year, about 0.9% per year to about 0.6% per year, about 0.9% per year to about 0.5% per year, about 0.9% per year to about 0.4% per year, about 0.9% per year to about 0.3% per year, about 0.9% per year to about 0.2% per year, about 0.9% per year to about 0.1% per year, about 0.9% per year to about 0.05% per year, about 0.8% per year to about 0.7% per year, about 0.8% per year to about 0.6% per year, about 0.8% per year to about 0.5% per year, about 0.8% per year to about 0.4% per year, about 0.8% per year to about 0.3% per year, about 0.8% per year to about 0.2% per year, about 0.8% per year to about 0.1% per year, about 0.8% per year to about 0.05% per year, about 0.7% per year to about 0.6% per year, about 0.7% per year to about 0.5% per year, about 0.7% per year to about 0.4% per year, about 0.7% per year to about 0.3% per year, about 0.7% per year to about 0.2% per year, about 0.7% per year to about 0.1% per year, about 0.7% per year to about 0.05% per year, about 0.6% per year to about 0.5% per year, about 0.6% per year to about 0.4% per year, about 0.6% per year to about 0.3% per year, about 0.6% per year to about 0.2% per year, about 0.6% per year to about 0.1% per year, about 0.6% per year to about 0.05% per year, about 0.5% per year to about 0.4% per year, about 0.5% per year to about 0.3% per year, about 0.5% per year to about 0.2% per year, about 0.5% per year to about 0.1% per year, about 0.5% per year to about 0.05% per year, about 0.4% per year to about 0.3% per year, about 0.4% per year to about 0.2% per year, about 0.4% per year to about 0.1% per year, about 0.4% per year to about 0.05% per year, about 0.3% per year to about 0.2% per year, about 0.3% per year to about 0.1% per year, about 0.3% per year to about 0.05% per year, about 0.2% per year to about 0.1% per year, about 0.2% per year to about 0.05% per year, or about 0.1% per year to about 0.05% per year. In some embodiments, degradation rate of the preserved nucleic acids is about 2% per year, about 1% per year, about 0.9% per year, about 0.8% per year, about 0.7% per year, about 0.6% per year, about 0.5% per year, about 0.4% per year, about 0.3% per year, about 0.2% per year, about 0.1% per year, or about 0.05% per year. In some embodiments, degradation rate of the preserved nucleic acids is at least about 2% per year, about 1% per year, about 0.9% per year, about 0.8% per year, about 0.7% per year, about 0.6% per year, about 0.5% per year, about 0.4% per year, about 0.3% per year, about 0.2% per year, or about 0.1% per year. In some embodiments, degradation rate of the preserved nucleic acids is at most about 1% per year, about 0.9% per year, about 0.8% per year, about 0.7% per year, about 0.6% per year, about 0.5% per year, about 0.4% per year, about 0.3% per year, about 0.2% per year, about 0.1% per year, or about 0.05% per year.

In some embodiments, the substrates comprising nucleic acid molecules are stored in one or more data centers. In some embodiments, the one or more data centers comprise a plurality of mountable racks configured to contain and maintain the substrates. In some embodiments, the one or more data centers comprise one or more instruments for sequencing nucleic acid molecules (sequencing by synthesis or other next generation sequencing techniques or other nucleic acid molecule sequencing techniques). In some embodiments, the instruments for sequencing nucleic acid molecules are configured to be rack mountable. In some embodiments, the one or more data centers are configured to support fully automated substrate storage and delivery to instruments for sequencing nucleic acid molecules.

In some embodiments, the systems and methods described herein reduce latency of retrieving the stored information (data request to delivery). In some embodiments, the time period for data retrieval is reduced to about 1 hour to about 12 hours. In some embodiments, the time period for data retrieval is reduced to about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 1 hour to about 11 hours, about 1 hour to about 12 hours, about 2 hours to about 3 hours, about 2 hours to about 4 hours, about 2 hours to about 5 hours, about 2 hours to about 6 hours, about 2 hours to about 7 hours, about 2 hours to about 8 hours, about 2 hours to about 9 hours, about 2 hours to about 10 hours, about 2 hours to about 11 hours, about 2 hours to about 12 hours, about 3 hours to about 4 hours, about 3 hours to about 5 hours, about 3 hours to about 6 hours, about 3 hours to about 7 hours, about 3 hours to about 8 hours, about 3 hours to about 9 hours, about 3 hours to about 10 hours, about 3 hours to about 11 hours, about 3 hours to about 12 hours, about 4 hours to about 5 hours, about 4 hours to about 6 hours, about 4 hours to about 7 hours, about 4 hours to about 8 hours, about 4 hours to about 9 hours, about 4 hours to about 10 hours, about 4 hours to about 11 hours, about 4 hours to about 12 hours, about 5 hours to about 6 hours, about 5 hours to about 7 hours, about 5 hours to about 8 hours, about 5 hours to about 9 hours, about 5 hours to about 10 hours, about 5 hours to about 11 hours, about 5 hours to about 12 hours, about 6 hours to about 7 hours, about 6 hours to about 8 hours, about 6 hours to about 9 hours, about 6 hours to about 10 hours, about 6 hours to about 11 hours, about 6 hours to about 12 hours, about 7 hours to about 8 hours, about 7 hours to about 9 hours, about 7 hours to about 10 hours, about 7 hours to about 11 hours, about 7 hours to about 12 hours, about 8 hours to about 9 hours, about 8 hours to about 10 hours, about 8 hours to about 11 hours, about 8 hours to about 12 hours, about 9 hours to about 10 hours, about 9 hours to about 11 hours, about 9 hours to about 12 hours, about 10 hours to about 11 hours, about 10 hours to about 12 hours, or about 11 hours to about 12 hours. In some embodiments, the time period for data retrieval is reduced to about 1 hour, about 2 hours, about 3 hours, about 4 hours, about 5 hours, about 6 hours, about 7 hours, about 8 hours, about 9 hours, about 10 hours, about 11 hours, or about 12 hours. In some embodiments, the time period for data retrieval is reduced to at least about 1 hour, about 2 hours, about 3 hours, about 4 hours, about 5 hours, about 6 hours, about 7 hours, about 8 hours, about 9 hours, about 10 hours, or about 11 hours. In some embodiments, the time period for data retrieval is reduced to at most about 2 hours, about 3 hours, about 4 hours, about 5 hours, about 6 hours, about 7 hours, about 8 hours, about 9 hours, about 10 hours, about 11 hours, or about 12 hours.

### Information Retrieval

One advantage of the data storage systems and methods described herein is, once the nucleic acid molecules and substrates are processed (disposed and preserved) by the systems and methods described herein, retrieval of the stored data requires little-to-no sample prep (e.g. amplification). In some embodiments, sample prep comprises disposing nucleic acids on to a substrate. In some embodiments, sample prep comprises amplification of nucleic acid molecules. In some embodiments, sample prep comprises polymerase chain reaction amplification. In some embodiments, sample prep comprises exposing the nucleic acid molecules to reagents appropriate for sequencing (sequencing by synthesis or other next generation sequencing techniques or other nucleic acid molecule sequencing techniques). As described herein, the nucleic acid molecules encoding particular information of interest are amplified prior to long-term storage. Thus, when informational retrieval is desired, the stored, amplified nucleic acid molecules merely need to be re-hydrated (if long-term storage techniques comprised lyophilization) and contacted with the appropriate nucleic acid extension reaction primers specific to the header sequence(s) corresponding to the sequences encoding the desired information to be retrieved.

In some embodiments, when utilizing the systems and methods described herein, the requirement of reagents appropriate for sequencing is reduced, as compared to the reagent requirement of current nucleic acid molecule sequencing systems and methods (e.g. current sequencing systems and methods utilized by Illumina®, Complete Genomics®, BGI®, or another nucleic acid sequencing company) by about 1× to about 12×. In some embodiments, when utilizing the systems and methods described herein, the requirement of reagents appropriate for sequencing is reduced by about 1× to about 2×, about 1× to about 3×, about 1× to about 4×, about 1× to about 5×, about 1× to about 6×, about 1× to about 7×, about 1× to about 8×, about 1× to about 9×, about 1× to about 10×, about 1× to about 11×, about 1× to about 12×, about 2× to about 3×, about 2× to about 4×, about 2× to about 5×, about 2× to about 6×, about 2× to about 7×, about 2× to about 8×, about 2× to about 9×, about 2× to about 10×, about 2× to about 11×, about 2× to about 12×, about 3× to about 4×, about 3× to about 5×, about 3× to about 6×, about 3× to about 7×, about 3× to about 8×, about 3× to about 9×, about 3× to about 10×, about 3× to about 11×, about 3× to about 12×, about 4× to about 5×, about 4× to about 6×, about 4× to about 7×, about 4× to about 8×, about 4× to about 9×, about 4× to about 10×, about 4× to about 11×, about 4× to about 12×, about 5× to about 6×, about 5× to about 7×, about 5× to about 8×, about 5× to about 9×, about 5× to about 10×, about 5× to about 11×, about 5× to about 12×, about 6× to about 7×, about 6× to about 8×, about 6× to about 9×, about 6× to about 10×, about 6× to about 11×, about 6× to about 12×, about 7× to about 8×, about 7× to about 9×, about 7× to about 10×, about 7× to about 11×, about 7× to about 12×, about 8× to about 9×, about 8× to about 10×, about 8× to about 11×, about 8× to about 12×, about 9× to about 10×, about 9× to about 11×, about 9× to about 12×, about 10× to about 11×, about 10× to about 12×, or about 11× to about 12×. In some embodiments, when utilizing the systems and methods described herein, the requirement of reagents appropriate for sequencing is reduced by about 1×, about 2×, about 3×, about 4×, about 5×, about 6×, about 7×, about 8×, about 9×, about 10×, about 11×, or about 12×. In some embodiments, when utilizing the systems and methods described herein, the requirement of reagents appropriate for sequencing is reduced by at least about 1×, about 2×, about 3×, about 4×, about 5×, about 6×, about 7×, about 8×, about 9×, about 10×, or about 11×. In some embodiments, when utilizing the systems and methods described herein, the requirement of reagents appropriate for sequencing is reduced by at most about 2×, about 3×, about 4×, about 5×, about 6×, about 7×, about 8×, about 9×, about 10×, about 11×, or about 12×.

In some embodiments, retrieval or reading of the stored information is possible after re-hydration of the nucleic acid molecules and/or substrates. In some embodiments, the retrieval or reading of the stored information comprises sequencing and detecting the nucleic acid molecules (as per US20150330974 or US20160201119 and/or U.S. Pat. No. 10,378,053).

Provided herein are systems and methods to facilitate imaging of signals from analytes immobilized or disposed on a surface with a center-to-center spacing below the diffraction limit (e.g. less than=λ/2*NA). These systems and methods use advanced imaging systems to generate high resolution images, and cycled detection to facilitate positional determination of molecules on the substrate with high accuracy and deconvolution of images to obtain signal identity for each molecule on a densely packed surface with high accuracy. These methods and systems allow single molecule sequencing by synthesis on a densely packed substrate to provide highly efficient and very high throughput polynucleotide sequence determination with high accuracy.

To achieve reduction in data storage costs, provided herein are methods and systems that facilitate reliable sequencing of polynucleotides immobilized or disposed on the surface of a substrate at a density below the diffraction limit. These high density arrays allow more efficient usage of reagents and increase the amount of data per unit area. In addition, the increase in the reliability of detection allows for a decrease in the number of clonal copies that must be synthesized to identify and correct errors in sequencing and detection, further reducing reagent costs and data processing costs.

### High Density Distributions of Analytes on a Surface of a Substrate

In a comparison of the proposed pitch compared to a sample effective pitch used for a $1,000 genome, the density of the new array is 170 fold higher, meeting the criteria of achieving 100 fold higher density. The number of copies per imaging spot per unit area also meets the criteria of being at least 100 fold lower than the prior existing platform. This helps ensure that the reagent costs are 100 fold more cost effective than baseline.

### Imaging Densely Packed Single Biomolecules and the Diffraction Limit

The primary constraint for increased molecular density for an imaging platform is the diffraction limit. The equation for the diffraction limit of an optical system is:

D=λ/2*NA

where D is the diffraction limit, λ is the wavelength of light, and NA is the numerical aperture of the optical system. Typical air imaging systems have NA's of 0.6 to 0.8. Using λ=600 nm, the diffraction limit is between 375 nm and 500 nm. For a water immersion system, the NA is ^{˜}1.0, giving a diffraction limit of 300 nm.

If features on an array or other substrate surface comprising biomolecules are too close, two optical signals will overlap so substantially so you just see a single blob that cannot be reliably resolved based on the image alone. This can be exacerbated by errors introduced by the optical imaging system, such as blur due to inaccurate tracking of a moving substrate, or optical variations in the light path between the sensor and the surface of a substrate.

The transmitted light or fluorescence emission wavefronts emanating from a point in the specimen plane of the microscope become diffracted at the edges of the objective aperture, effectively spreading the wavefronts to produce an image of the point source that is broadened into a diffraction pattern having a central disk of finite, but larger size than the original point. Therefore, due to diffraction of light, the image of a specimen never perfectly represents the real details present in the specimen because there is a lower limit below which the microscope optical system cannot resolve structural details.

The observation of sub-wavelength structures with microscopes is difficult because of the diffraction limit. A point object in a microscope, such as a fluorescent protein or nucleotide single molecule, generates an image at the intermediate plane that consists of a diffraction pattern created by the action of interference. When highly magnified, the diffraction pattern of the point object is observed to consist of a central spot (diffraction disk) surrounded by a series of diffraction rings. Combined, this point source diffraction pattern is referred to as an Airy disk.

The size of the central spot in the Airy pattern is related to the wavelength of light and the aperture angle of the objective. For a microscope objective, the aperture angle is described by the numerical aperture (NA), which includes the term sin θ, the half angle over which the objective can gather light from the specimen. In terms of resolution, the radius of the diffraction Airy disk in the lateral (x,y) image plane is defined by the following formula: Abbe Resolution x,y=λ/2*NA, where λ is the average wavelength of illumination in transmitted light or the excitation wavelength band in fluorescence. The objective numerical aperture (NA=n·sin(θ)) is defined by the refractive index of the imaging medium (n; usually air, water, glycerin, or oil) multiplied by the sine of the aperture angle (sin(θ)). As a result of this relationship, the size of the spot created by a point source decreases with decreasing wavelength and increasing numerical aperture, but always remains a disk of finite diameter. The Abbe resolution (i.e., Abbe limit) is also referred to herein as the diffraction limit and defines the resolution limit of the optical system.

If the distance between the two Airy disks or point-spread functions is greater than this value, the two point sources are considered to be resolved (and can readily be distinguished). Otherwise, the Airy disks merge together and are considered not to be resolved.

Thus, light emitted from a single molecule detectable label point source with wavelength λ, traveling in a medium with refractive index n and converging to a spot with half-angle θ will make a diffraction limited spot with a diameter: d=λ/2*NA. Considering green light around 500 nm and a NA (Numerical Aperture) of 1, the diffraction limit is roughly d=λ/232 250 nm (0.25 μm), which limits the density of analytes such as single molecule proteins and nucleotides on a surface able to be imaged by conventional imaging techniques. Even in cases where an optical microscope is equipped with the highest available quality of lens elements, is perfectly aligned, and has the highest numerical aperture, the resolution remains limited to approximately half the wavelength of light in the best case scenario.

### Deconvolution

Deconvolution is an algorithm-based process used to reverse the effects of convolution on recorded data. The concept of deconvolution is widely used in the techniques of signal processing and image processing. Because these techniques are in turn widely used in many scientific and engineering disciplines, deconvolution finds many applications.

In optics and imaging, the term “deconvolution” is specifically used to refer to the process of reversing the optical distortion that takes place in an optical microscope, electron microscope, telescope, or other imaging instrument, thus creating clearer images. It is usually done in the digital domain by a software algorithm, as part of a suite of microscope image processing techniques.

The usual method is to assume that the optical path through the instrument is optically perfect, convolved with a point spread function (PSF), that is, a mathematical function that describes the distortion in terms of the pathway a theoretical point source of light (or other waves) takes through the instrument. Usually, such a point source contributes a small area of fuzziness to the final image. If this function can be determined, it is then a matter of computing its inverse or complementary function, and convolving the acquired image with that. Deconvolution maps to division in the Fourier co-domain. This allows deconvolution to be easily applied with experimental data that are subject to a Fourier transform. An example is NMR spectroscopy where the data are recorded in the time domain, but analyzed in the frequency domain. Division of the time-domain data by an exponential function has the effect of reducing the width of Lorenzian lines in the frequency domain. The result is the original, undistorted image.

However, for diffraction limited imaging, deconvolution is also needed to further refine the signals to improve resolution beyond the diffraction limit, even if the point spread function is known. It is very hard to separate two objects reliably at distances smaller than the Nyquist distance. However, described herein are methods and systems using cycled detection, analyte position determination, alignment, and deconvolution to reliably detect objects separated by distances much smaller than the Nyquist distance.

### Sequencing

Optical detection imaging systems are diffraction-limited, and thus have a theoretical maximum resolution of ^{˜}300 nm with fluorophores typically used in sequencing. To date, the best sequencing Systems have had center-to-center spacings between adjacent polynucleotides of ^{˜}600 nm on their arrays, or ^{˜}2×the diffraction limit. This factor of 2× is needed to account for intensity, array & biology variations that can result in errors in position. For sequencing, the purpose of the system and methods described herein are to resolve polynucleotides that are sequenced on a substrate with a center-to-center spacing below the diffraction limit of the optical system.

As described herein, we provide methods and systems to achieve sub-diffraction-limited imaging in part by identifying a position of each analyte with a high accuracy (e.g., 10 nm RMS or less). By comparison, state of the art Super Resolution systems (Harvard/STORM) can only identify location with an accuracy down to 20 nm RMS, 2× worse than this system. Thus, the methods and system disclosed herein enable sub-diffraction limited-imaging to identify densely-packed molecules on a substrate to achieve a high data rate per unit of enzyme, data rate per unit of time, and high data accuracy. These sub-diffraction limited imaging techniques are broadly applicable to techniques using cycled detection as described herein.

### Imaging and Cycled Detection

As described herein, each of the detection methods and systems required cycled detection to achieve sub-diffraction limited imaging. Cycled detection includes the binding and imaging or probes, such as antibodies or nucleotides, bound to detectable labels that are capable of emitting a visible light optical signal. By using positional information from a series of images of a field from different cycles, deconvolution to resolve signals from densely packed substrates can be used effectively to identify individual optical signals from signals obscured due to the diffraction limit of optical imaging. After multiple cycles the precise location of the molecule will become increasingly more accurate. Using this information, additional calculations can be performed to aid in crosstalk correction regarding known asymmetries in the crosstalk matrix occurring due to pixel discretization effects.

Methods and systems using cycled probe binding and optical detection are described in US Publication No. 2015/0330974, Digital Analysis of Molecular Analytes Using Single Molecule Detection, published Nov. 19, 2015, which is incorporated herein by reference in its entirety.

In some embodiments, the raw images are obtained using sampling that is at least at the Nyquist limit to facilitate more accurate determination of the oversampled image. Increasing the number of pixels used to represent the image by sampling in excess of the Nyquist limit (oversampling) increases the pixel data available for image processing and display.

Theoretically, a bandwidth-limited signal can be perfectly reconstructed if sampled at the

Nyquist rate or above it. The Nyquist rate is defined as twice the highest frequency component in the signal. Oversampling improves resolution, reduces noise and helps avoid aliasing and phase distortion by relaxing anti-aliasing filter performance requirements. A signal may be oversampled by a factor of N if it is sampled at N times the Nyquist rate.

Thus, in some embodiments, each image is taken with a pixel size no more than half the wavelength of light being observed. Put in another way, a wavelength of a signal generated from one or more detectable labels detected on an optical detection system is greater than two times a pixel of the optical detection system. For example, in some embodiments, a pixel size of 162.5 nm×162.5 nm is used in detection to achieve sampling at or above the Nyquist limit. Sampling at a frequency of at least the Nyquist limit during raw imaging of the substrate is preferred to optimize the resolution of the system or methods described herein. This can be done in conjunction with the deconvolution methods and optical systems described herein to resolve features on a substrate below the diffraction limit with high accuracy.

### Error-Correction Methods

In optical and electrical detection methods described above, errors can occur in binding and/or detection of signals. In some cases, the error rate can be as high as one in five (e.g., one out of five fluorescent signals is incorrect). This equates to one error in every five-cycle sequence. Actual error rates may not be as high as 20%, but error rates of a few percent are possible. In general, the error rate depends on many factors including the type of analytes in the sample and the type of probes used. In an electrical detection method, for example, a tail region may not properly bind to the corresponding probe region on an aptamer during a cycle. In an optical detection method, an antibody probe may not bind to its target or bind to the wrong target.

Additional cycles are generated to account for errors in the detected signals and to obtain additional bits of information, such as parity bits. The additional bits of information are used to correct errors using an error correcting code. In one embodiment, the error correcting code is a Reed-Solomon code, which is a non-binary cyclic code used to detect and correct errors in a system. In other embodiments, various other error correcting codes can be used. Other error correcting codes include, for example, block codes, convolution codes, Golay codes, Hamming codes, BCH codes, AN codes, Reed-Muller codes, Goppa codes, Hadamard codes, Walsh codes, Hagelbarger codes, polar codes, repetition codes, repeat-accumulate codes, erasure codes, online codes, group codes, expander codes, constant-weight codes, tornado codes, low-density parity check codes, maximum distance codes, burst error codes, luby transform codes, fountain codes, and raptor codes. See Error Control Coding, 2nd Ed., S. Lin and D J Costello, Prentice Hall, N.Y., 2004. Examples are also provided below that demonstrate the method for error-correction by adding cycles and obtaining additional bits of information.

### Optical Detection Methods

In some embodiments, a substrate is bound with analytes comprising N target analytes.

To detect N target analytes, M cycles of probe binding and signal detection are chosen. Each of the M cycles includes 1 or more passes, and each pass includes N sets of probes, such that each set of probes specifically binds to one of the N target analytes. In certain embodiments, there are N sets of probes for the N target analytes.

In each cycle, there is a predetermined order for introducing the sets of probes for each pass. In some embodiments, the predetermined order for the sets of probes is a randomized order. In other embodiments, the predetermined order for the sets of probes is a non-randomized order. In one embodiment, the non-random order can be chosen by a computer processor. The predetermined order is represented in a key for each target analyte. A key is generated that includes the order of the sets of probes, and the order of the probes is digitized in a code to identify each of the target analytes.

In some embodiments, each set of ordered probes is associated with a distinct tag for detecting the target analyte, and the number of distinct tags is less than the number of N target analytes. In that case, each N target analyte is matched with a sequence of M tags for the M cycles. The ordered sequence of tags is associated with the target analyte as an identifying code.

In one embodiment, the method includes the following steps for labeling probe pools to count N different kinds of target analytes on a substrate using fluorescently tagged probes of X different colors:
1. Number a list of the N targets (or their probes) using base-X numbers.
2. Associate fluorescent tags with base-X digits from 0 to X−1. (For example, 0, 1, 2, 3 correspond to red, blue, green, yellow.)
3. Find C such that XC>N.
4. At least C probe pools are needed to identify the N targets. Label the C probe pools by an index k=1 to C.
5. In the kth probe pool, label each probe with a fluorescent tag of the color that corresponds to the kth base-X digit of the base-X number that identifies the probe's target in the list created in Step 1.

For example, if one has N=10,000 target analytes and four fluorescent tags, a base 4 can be chosen. The 4 fluorescent tag colors designated with the numbers 0, 1, 2, and 3, respectively. For example, numbers 0, 1, 2, 3 correspond to red, blue, green, and yellow.

When base 4 is chosen, each fluorescent color is represented by 2 bits (0 and 1, where 0=no signal and 1=signal present), and there are 7 colors that are used as a code to identify a target analyte. For example, protein A may be identified with the code of “1221133” that represents the color combination and order of “blue, green, green, blue, blue, yellow, yellow.” For the 7 possible colors, there are a total of 14 bits of information for the target analyte (7×2=14 bits).

Next, C is chosen such that 4C>10,000. In this case, C can be 7 such that there are 7 probe pools to identify 10,000 targets (47=16,384, which is greater than 10,000). A color sequence of length C means that C different probe pools must be constructed. The 7 probe pools are labeled from k=1 to 7. Then each probe is labeled with a fluorescent tag that corresponds to the kth base and X-digit. For example, the third probe in the code “1221133” will be the 3rd base-4th digit and corresponds to green.

### Quantification of Optically-Detected Probes

After the detection process, the signals from each probe pool are counted, and the presence or absence of a signal and the color of the signal can be recorded for each position on the substrate.

From the detectable signals, K bits of information are obtained in each of M cycles for the N distinct target analytes. The K bits of information are used to determine L total bits of information, such that K×M=L bits of information and L≥log 2 (N). The L bits of information are used to determine the identity (and presence) of N distinct target analytes. If only one cycle (M=1) is performed, then K×1=L. However, multiple cycles (M>1) can be performed to generate more total bits of information L per analyte. Each subsequent cycle provides additional optical signal information that is used to identify the target analyte.

In practice, errors in the signals occur, and this confounds the accuracy of the identification of target analytes. For instance, probes may bind the wrong targets (e.g., false positives) or fail to bind the correct targets (e.g., false negatives). Methods are provided, as described below, to account for errors in optical and electrical signal detection.

### Electrical Detection Methods

In other embodiments, electrical detection methods are used to detect the presence of target analytes on a substrate. Target analytes are tagged with oligonucleotide tail regions and the oligonucleotide tags are detected using ion-sensitive field-effect transistors (ISFET, or a pH sensor), which measures hydrogen ion concentrations in solution.

ISFETs present a sensitive and specific electrical detection system for the identification and characterization of analytes. In one embodiment, the electrical detection methods disclosed herein are carried out by a computer (e.g., a processor). The ionic concentration of a solution can be converted to a logarithmic electrical potential by an electrode of an ISFET, and the electrical output signal can be detected and measured.

ISFETs have previously been used to facilitate DNA sequencing. During the enzymatic conversion of single-stranded DNA into double-stranded DNA, hydrogen ions are released as each nucleotide is added to the DNA molecule. An ISFET detects these released hydrogen ions and can determine when a nucleotide has been added to the DNA molecule. By synchronizing the incorporation of the nucleoside triphosphates (dATP, dCTP, dGTP, and dTTP), the DNA sequence may also be determined. For example, if no electrical output signal is detected when the single-stranded DNA template is exposed to dATP's, but an electrical output signal is detected in the presence of dGTP's, the DNA sequence is composed of a complementary cytosine base at the position in question.

In one embodiment, an ISFET is used to detect a tail region of a probe and then identify corresponding target analyte. For example, a target analyte can be immobilized on a substrate, such as an integrated-circuit chip that contains one or more ISFETs. When the corresponding probe (e.g., aptamer and tail region) is added and specifically binds to the target analyte, nucleotides and enzymes (polymerase) are added for transcription of the tail region. The ISFET detects the release hydrogen ions as electrical output signals and measures the change in ion concentration when the dNTP's are incorporated into the tail region. The amount of hydrogen ions released corresponds to the lengths and stops of the tail region, and this information about the tail regions can be used to differentiate among various tags.

The simplest type of tail region is one composed entirely of one homopolymeric base region. In this case, there are four possible tail regions: a poly-A tail, a poly-C tail, a poly-G tail, and a poly-T tail. However, it is often desirable to have a great diversity in tail regions.

One method of generating diversity in tail regions is by providing stop bases within a homopolymeric base region of a tail region. A stop base is a portion of a tail region comprising at least one nucleotide adjacent to a homopolymeric base region, such that the at least one nucleotide is composed of a base that is distinct from the bases within the homopolymeric base region. In one embodiment, the stop base is one nucleotide. In other embodiments, the stop base comprises a plurality of nucleotides. Generally, the stop base is flanked by two homopolymeric base regions. In an embodiment, the two homopolymeric base regions flanking a stop base are composed of the same base. In another embodiment, the two homopolymeric base regions are composed of two different bases. In another embodiment, the tail region contains more than one stop base.

In one example, an ISFET can detect a minimum threshold number of 100 hydrogen ions. Target Analyte 1 is bound to a composition with a tail region composed of a 100-nucleotide poly-A tail, followed by one cytosine base, followed by another 100-nucleotide poly-A tail, for a tail region length total of 201 nucleotides. Target Analyte 2 is bound to a composition with a tail region composed of a 200-nucleotide poly-A tail. Upon the addition of dTTP's and under conditions conducive to polynucleotide synthesis, synthesis on the tail region associated with Target Analyte 1 will release 100 hydrogen ions, which can be distinguished from polynucleotide synthesis on the tail region associated with Target Analyte 2, which will release 200 hydrogen ions. The ISFET will detect a different electrical output signal for each tail region. Furthermore, if dGTP's are added, followed by more dTTP's, the tail region associated with Target Analyte 1 will then release one, then 100 more hydrogen ions due to further polynucleotide synthesis. The distinct electrical output signals generated from the addition of specific nucleoside triphosphates based on tail region compositions allow the ISFET to detect hydrogen ions from each of the tail regions, and that information can be used to identify the tail regions and their corresponding target analytes.

Various lengths of the homopolymeric base regions, stop bases, and combinations thereof can be used to uniquely tag each analyte in a sample. Additional description about electrical detection of aptamers and tail regions to identify target analytes in a substrate are described in U.S. Patent Application No. 2016/0201119, which is incorporated by reference in its entirety.

In some embodiments, the large amount of information in the stored data catalogue on the substrate(s) generates several levels of built-in redundancy. In some embodiments, the first level of information subdivision is comprised in the slide, lane and specific sequencing priming site for each information segment of data. In some embodiments, the individual lanes are stored in various combinations that are generated to be optimum for retrieval as described herein.

### Computer-Automation of the Systems and Methods Described Herein

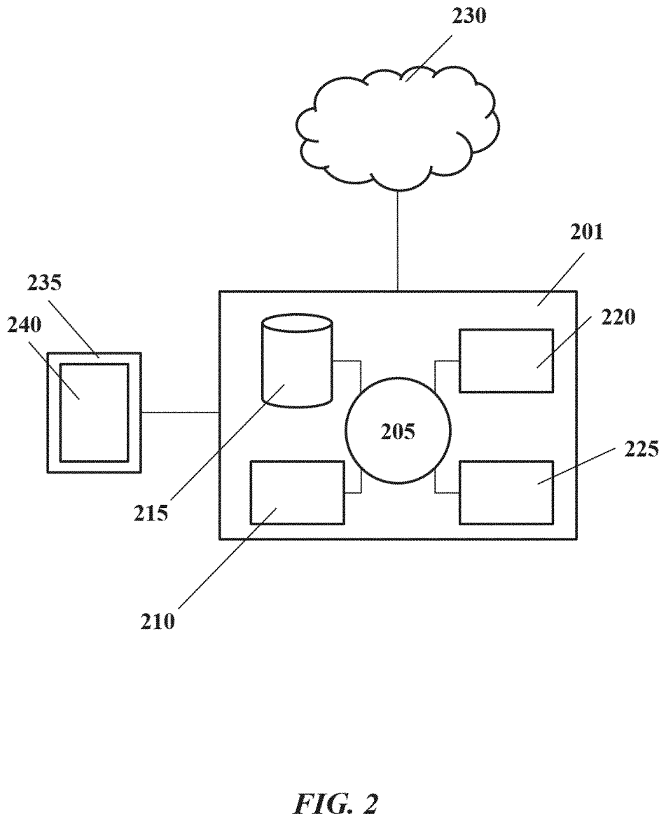

The present disclosure provides computer systems that are programmed to implement methods of the disclosure. FIG. 2 shows a computer system 201 that is programmed or otherwise configured to dispose the substrates onto mountable racks within a data center and retrieve and deliver the substrates to instruments also contained within the data centers for sequencing. The computer system 201 can regulate various aspects of the present disclosure, such as, for example, the temperature of the data center and the configuration of the substrates stored within the data center. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server. In some embodiments, the network 230, comprises instruments for mechanically transporting substrates to mountable storage racks and to instruments for sequencing. In some embodiments, the network 230, comprises instruments for sequencing.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs and nucleic acid sequencing read-outs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., an instrument for sequencing). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, the results of nucleic acid molecule sequencing. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, generate a rate for which substrates are transported to and from the mountable racks for storage and instruments for sequencing.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Methods and systems provided herein may be combined with or modified by other methods and systems, such as, for example, those described in U.S. Patent Publication Nos. 20150330974 and 20180274028, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-73. (canceled)

74. A method for storing data, comprising:
(a) encoding said data in a nucleic acid sequence;
(b) generating one or more linear nucleic acid molecules comprising at least a portion of said. nucleic acid sequences and circularizing said one or more linear nucleic acid molecules and amplifying by rolling circle amplification to generate one or more concatenated nucleic acid molecules; and
(c) storing said one or more linear nucleic acid molecules in an array disposed on a substrate, to provide said array when said array is imaged using an optical scanning system, wherein a wavelength of a signal generated from said one or more linear nuclei acid molecules or derivative thereof is greater than two times a size of a pixel of said optical scanning system.

75. The method of claim 74, wherein (b) comprises:
(a) generating one or more linear nucleic acid molecules that comprise said nucleic acid. sequence, a first adapter sequence, and a second adapter sequence, wherein said first and said second adapter sequence enable formation of one or more circular nucleic acid molecules; and
(b) amplifying said one or more circular nucleic acid molecules.

76. The method of claim 75, wherein said linear nucleic acid molecule comprises one or more functional sequences.

77. The method of claim 75, wherein said one or more circular nucleic acid molecules are generated by rolling circle amplification.

78. The method of claim 77, wherein (c) comprises disposing said one or more circular nucleic on said substrate.

79. The method of claim 78, wherein said one or more circular nucleic acid molecules are disposed at a density wherein an average distance between two or more nucleic acid molecules is less than a measure of λ/(2*NA), wherein λ is said wavelength of said signal generated from said one or more linear nucleic acids or derivatives thereof, and wherein NA is a numerical aperture of said optical scanning system.

80. The method of claim 74, further comprising retrieving said data from said one or more linear nucleic acid molecules without amplification prior to said retrieving.

81. The method of claim 80, wherein said retrieving comprises sequencing said one or more linear nucleic acid molecules or derivatives thereof.

82. The method of claim 81, wherein said sequencing comprises detecting one or more incorporated nucleic acids using said optical scanning system.

83. A method for storing data, comprising disposing a nucleic acid molecule to a substrate, wherein said nucleic molecule or derivative thereof encodes said data, and wherein said data is retrieved from said nucleic acid molecule without amplification prior to sequencing.

84. The method of claim 83, wherein said nucleic acid molecule or derivative thereof comprises a nucleic acid concatemer.

85. The method of claim 83, wherein said nucleic acid molecule or derivative thereof is disposed at a density wherein when said substrate is imaged using an optical scanning system, a wavelength of a signal generated from said nucleic acid molecule or derivative thereof is greater than two times a size of a pixel of said optical scanning system.

86. The method of claim 83, wherein said substrate comprises silicon.

87. The method of claim 83, wherein said substrate comprises glass.

88. The method of claim 83, wherein said substrate comprises two pieces of glass.

89. The method of claim 83, wherein said nucleic acid molecule comprises a linear nucleic acid molecule.

90. The method of claim 89, wherein said linear nucleic acid molecule comprises one or more functional sequences.

91. The method of claim 83, wherein said data is retrieved by sequencing said one or more nucleic acid molecules or derivatives thereof.

92. The method of claim 91, wherein said sequencing comprises detecting one or more incorporate nucleic acids using a detection system.

93. The method of claim 92, wherein said detection system comprises an optical scanning system.
